(19) United States
(12) Patent Application Publication
(10) Pub. No.: US 2003/0100938 A1
(43) Pub. Date: May 29, 2003

(54) In-stent restenosis detection device

(75) Inventors: Alexander M. Rubenchik, Livermore, CA (US); Gilbert W. Collins, Livermore, CA (US); Joseph D. Kilkenny, Livermore, CA (US); Paul J. Weber, Fort Lauderdale, FL (US); Luiz B. Da Silva, Danville, CA (US)

Correspondence Address: John P. Wooldridge, 1334 Ridgestone Court, Livermore, CA, 94550, US

(73) Assignee: Pearl Technology Holdings II, LLC

(21) Appl. No.: 09/995,284

(22) Filed: Nov. 27, 2001

(51) Int. Cl. A61M 37/00; A61B 17/52; A61N 2/00; A61F 2/06

(52) U.S. Cl. 623/1.1; 600/9; 600/12

(57) ABSTRACT

A medical device is described that can be used to detect in stent restenosis. This invention consists of a stem of current or modified technology and a microwave transmitter/receiver to detect stent performance in-situ.

### BACKGROUND OF THE INVENTION

1. Field of the Invention

The present invention relates to a medical device that can be used to evaluate in-stent restenosis. The present invention also relates to a microwave device that can be used to monitor occlusions inside implanted stents.

2. Description of Related Art

An estimated seven million Americans suffer from coronary artery disease, which causes 1.5 million myocardial infarctions (heart attacks) and over half a million deaths annually at a cost of over $100 billion. Coronary artery disease results from atherosclerosis, a complex process in which fatty and other deposits (e.g., cellular intimal and mineral additives, and engrained proteinaceous or clotting/platelet debris) build up on the walls of arteries, resulting in blockages and reduced blood flow. This process leads to the formation of a plaque of atherosclerotic material that can be comprised of various cells, lipids (fats or cholesterol), and collagen (fibrous tissue). This process progresses over a number of years and may eventually result in the formation of a blockage (stenosis) in the coronary artery. If the artery is sufficiently narrowed, blood flow is reduced (ischemia), and chest pain (angina pectoris), heart attack, or sudden death may follow. In addition to the narrowing produced by atherosclerosis, plaques may also rupture, resulting in the formation of a thrombus (clot) on the plaque surface, leading to an abrupt cessation of blood flow to the heart. Plaque rupture plays a key role in most cases of heart attack and stroke.

In 1977, Dr. Andreas Gruentzig from Switzerland introduced a novel method for treating coronary artery stenosis, which he termed Percutaneous Transluminal Coronary Angioplasty (PTCA) also commonly known as balloon angioplasty. Over 500,000 coronary angioplasties (the term angioplasty is derived from angio, which refers to a blood vessel, and plasty, which means to reshape) were performed in the U.S., surpassing the number of coronary bypass operations. The advantage of this technique is that it can be performed using minimally invasive catheter procedures. Using special x-ray equipment and contrast dye to visualize the arteries, the cardiologist advances a guide catheter (hollow tube) through the sheath and up the aorta to the origin of the coronary arteries. Using this catheter as a track to the coronary artery, a long, fine guidewire (generally 0.014 inches in diameter) is negotiated across the stenosis. A catheter with a deflated balloon on the far end is then advanced over the guidewire to the narrowed arterial segment. At this point the balloon is inflated and the occluding plaque compressed to the arterial wall.

In conventional PTCA the occluding plaque is simply compressed and no material is removed. In about one-third of cases, re-narrowing of the treated segment may occur over a period of several months, necessitating a repeat procedure or coronary artery bypass surgery. This re-narrowing is termed restenosis and appears to be distinct from the process of atherosclerosis. Despite intense research efforts and numerous drug trials, a solution to this problem remains elusive.

In order to reduce the restenosis rate, stents are now routinely inserted into arteries after PTCA. Stents are wire mesh tubes usually made of metal that are expanded within the artery to form a scaffold that keeps the artery open. The stent stays in the artery permanently, holds it open, improves blood flow to the heart muscle and relieves symptoms (usually chest pain). However, even with stents, the restenosis rate can be as high as 25%.

Restenosis within the stent can be detected in several ways. If the patient is symptomatic with angina then the physician can perform coronary angiography. In this procedure a catheter is inserted into the patient and x-ray contrast agent injected so that the blood flow can be imaged with x-rays. Typically coronary angiography is performed 6 months after stent insertion, or earlier if symptoms or exercise tolerance tests suggested restenosis. This is an invasive procedure requiring the use of an operating room and exposes the patient to x-ray radiation. More recently, exercise stress tests are recommended to evaluate cardiac function and possible patient discomfort. If the patient fails the stress test then coronary angiography is performed. An alternative technique, exercise radionuclide ventriculography, is also used now to evaluate cardiac function and possible restenosis. Unfortunately, this procedure requires injecting radioactive markers into the patient and the use of expensive gamma imaging cameras and requires above 70% blockage of blood flow to give a positive result.

Another emerging technique is intravascular ultrasound where a miniature ultrasound transducer is inserted by a catheter and the acoustic impedance of the blood vessel is monitored by external acoustic receivers is also being evaluated for efficacy. Unfortunately this is of course an invasive procedure.

Recently Spillman et al. (WO 00/56210) and Cimochowski et al. (WO 99/26530) described a novel stent design that incorporates a miniature sensor that can be used to measure flow or pressure and diagnose restenosis. Unfortunately, these devices require incorporating a sensor into the stent that could adversely affect the mechanical properties of the stent.

Given the limitaions of current techniques to diagnose restenosis, there is a need for a novel device that can safely, quickly and effectively detect restenosis after stenting. The present invention fulfills this need, and further provides related advantages.

### SUMMARY OF THE INVENTION

An object of the present invention is to provide a device and method to evaluate in-stent restenosis.

Another object of the present invention is to provide an endoluminal implant (or stent) that can be inserted into the body and probed with high frequency (0.1 to 20 GHz) electromagnetic radiation (or microwaves) to determine a change in the aperture (i.e., lumen) for blood flow within the stent.

For treating coronary occlusions, the endoluminal implant is generally a tubular shaped member having at least two configurations. Initially it has a compact configuration in which the member has a cross-sectional size smaller than the lumen at the treatment area. After placement it has an expanded configuration in which the member has a cross-sectional size comparable to the lumen of the treatment area. The implant includes at least a portion that is made of an electrical conducting material.

The endoluminal implant system further includes an external microwave transmitter and receiver for probing the endoluminal implant. The microwave transmitter can generate a wide frequency range of microwaves (e.g., 0.1 to 20 GHz). The receiver detects the microwaves scattered, reflected and possibly transmitted from the endoluminal implant. The received amplitude as a function of microwave frequency and polarization can be analyzed to determine the conditions within and around the endoluminal implant

In one embodiment, the endoluminal implant is made of a high microwave scattering material (e.g., conductive metal). In another embodiment the geometrical structure of the implant is designed to scatter (and/or reflect) microwaves within a narrower frequency range or lower frequency or specific polarization state.

In normal use to treat and monitor coronary occlusions, a physician inserts the endoluminal implant (stent) into the patient using a balloon catheter. After the procedure is completed or, e.g., within about a month, the patient is placed in proximity to the microwave transmitter and receiver apparatus and a detailed measurement of the reflected and scattered microwave amplitude as a function of microwave frequency and polarization angle is performed. Typical stents are hollow metal wire mesh cylinders that can be approximated as a cylindrical waveguide. The microwave scattering cross-section on this cylinder has peaks at frequencies characteristic of the resonance frequencies of the waveguide. This data is collected and recorded to provide a baseline frequency response around the frequencies of natural resonance of the stent for the patient with no restenosis. In subsequent months the patient visits the doctor's office and a new frequency response is measured. The new frequency response is compared to the baseline frequency response and any previous measurements to identify possible restenosis. When the change in the frequency response exceeds some predetermined limit, the doctor can be advised to perform additional tests including angiography.

In an alternative embodiment of the device, the patient is provided with a compact device that can be used at home to measure the scattering amplitude over a narrow range of frequencies in the vicinity of the implant resonance. In the simplest form the compact device measures the scattered microwave amplitude at two frequencies one at the implant resonance and one off resonance. The off resonance amplitude can be used to normalize the scattered amplitude and correct differences in the positioning of the device from use to use. If the measured scattered resonance amplitude changes then it is likely due to plaque formation. In this case the patient can visit the doctor for a more detailed measurement over the full microwave frequency range.

A change in the resonance frequency of the implant occurs because the dielectric properties of plaque differ significantly from that of blood (for example near 10 GHz, the relative permittivity (dielectric constant) ε of blood is about 64 times larger than plaque).

There are several studies documenting the loss tangent versus frequency for tissue, blood, bone, etc. Typically the attenuation in the microwave range increases with increasing frequency from 0.1 to tens of GHz, and therefore optimal performance is achieved at the lowest eigen-frequency of the stent. The minimal H-mode eigen-frequency for a metal cylinder of radius a is given by

$$f = 1.84 \frac{c}{2 π a \sqrt{ϵ}} \qquad ( 1 )$$

Blood has a high relative permittivity ε and \sqrt{ε}˜8, for f<10 Ghz. Therefore, a blood filled cylinder with a=0.1 cm gives f˜11 GHz. As plaque or a thrombus forms on the stent, the dielectric constant inside the resonator changes by δε resulting in an increase in its resonance frequency. The frequency shift is given by

$$- \frac{δ f}{f} = \frac{{\int }_{}^{} {\vert E \vert}^{2} δ ϵ ⅆ V}{2 ϵ {\int }_{}^{} {\vert E \vert}^{2} ⅆ V} ∼ \frac{δ ϵ h}{ϵ a} \qquad ( 2 )$$

Here E is the electric field distribution for the lowest H mode and δε is the change of dielectric constant within the plaque and h is the thickness of the plaque. Given the difference between plaque and blood, frequency shifts of several percent are possible.

When plaque starts to form in the stents, the eigenfrequency of the stent will increase shifting the scattering peak position to higher frequencies. The amount of the frequency shift can be used to determine the extent of restenosis.

The exact value of the resonance-frequency will depend in detail on the exact shape of the stent and can be slightly different from (1) due to the environment and the finite size of the cylinder. The value of the scattering peak is determined by the convolution of the coupling of the incident electromagnetic wave with the field inside the cylinder and Eigen mode damping. For example, the up shift in frequency due to the finite length of the cylinder L is given by

$$- \frac{δ f}{f} = ( 1 + \frac{{k}_{z}^{2} {a}^{2}}{6.8} ) ∼ ( 1 + 1.5 \frac{{a}^{2}}{{L}^{2}} )$$

This shift is less than the shift expected from plaque build up within the stent as approximated by equation (2) and most importantly will not change with time.

In order to minimize the background signal due to reflection and scattering from the skin and other parts of the body, the intraluminal implant can be designed to have enhanced reflectivity at a specific polarization angle. For example using linearly polarized fields even for a simple conducting cylinder with the electric field polarized along the axis of the cylinder will generate current along the axis only and thus the scattered magnetic field intensity along the axis is zero. Similarly, an incident wave with its magnetic field vector polarized along the cylinder axis yields a scattered wave with no electric field component along the cylinder axis. Thus, simple linear polarization of the incident field yields large discrimination. Rotating the polarization and detecting versus phase rotation can also be used to increase signal to noise. A more general solution is provided for the case of an infinite cylinder and incident wave E-polarized along the axis to obtain the scattering cross section (defined as the total scattered power divided by the Poynting vector in 3D)

$${σ}_{sc} = \frac{4}{{k}^{2}} {\sum }_{m = - ∞}^{∞} \frac{{J}_{m}^{2} ( ka )}{{J}_{m}^{2} ( ka ) + {N}_{m}^{2} ( ka )}$$

whereas a B-polarized incident wave would produce

$${σ}_{sc} = \frac{4}{{k}^{2}} {\sum }_{m = - ∞}^{∞} \frac{{J}_{m}^{′2} ( ka )}{{J}_{m}^{′2} ( ka ) + {N}_{m}^{′2} ( ka )} .$$

where J is Bessel's function of the first kind, N is Neumann function, J′, N′ is the first derivative of J, N, k is the wave vector in the medium of propagation, and a is the radius of the cylinder.

Some embodiments will use stents currently used in the field without modification. However the 11 GHz resonance frequency of typical stents is in a region of the microwave spectrum where the absorption in tissue is quite high. And so in other embodiments, the material or geometry will be modified to enhance the microwave scattering or to lower the resonance frequency of the stent. Current stents such as that shown in FIG. 2 would function as described above. To enhance the Q or the signal produced through buildup of plaque, the stent geometry can be modified. As one example, the cylindrical symmetry of the stent can be broken. Even in these cases polarization will improve discrimination. FIG. 3 shows possible embodiments for scatterers with broken symmetry. In these cases the change in dielectric constant due to restenosis changes the resonant frequency in two ways. First the frequency changes due to the effect described above. In addition, because the E-field is concentrated at the gap, a very small change in dielectric constant near the gap (a very small degree of restenosis) can be detected. Alternatively the space between the shield and the stent can be filled with a high dielectric constant material to reduce the resonance frequency of the stent to the low GHz range where tissue absorption is lower (however the reflection at tissue interfaces is higher). Alternatively the stent can be constructed so that electrically it is a helical winding with n turns along its length. In this case the inductance of the stent increases approximately as n² and so the resonance frequency decreases approximately as 1/n.

In a further embodiment of the invention the space between the azimuthal break has a microwave diode inserted. The non-linear response with microwave amplitude which results from the non-linear impedance of the diode generates harmonics of the fundamental microwave frequency which will be easier to measure against the scattering background from tissue interfaces which will all be at the fundamental frequency of the microwaves.

The use of low microwave power and energy makes this device safe for the patient and eliminates the need for unnecessary x-ray imaging. To increase the signal to noise, the device can include an acoustic modulator or detect on the natural rhythmic variations of the patient such as heart beat rate. These slight modifications are easily adapted into the device.

Additional background signal rejection can be achieved by time gating the return microwave signal to eliminate reflected microwaves from outside the area of interest. For example, nanosecond pulses localize the detection to a few centimeters. For these embodiments, Q (which is related to the deadtime) will need to be low. While this is not a problem with some embodiments, others may need to have their Q spoiled or lowered through overcoupling. A very simple embodiment of a pulsed transmitter/receiver can be achieved through adding a pulse modulator and traveling wave tube on the output of the broad-band source, and switching on/off the detection/receiving arms to avoid saturation of the detectors during the microwave pulse.

If the stent is tuned with a high Q this device might allow destruction of plaque buildup through the efficient channeling of microwaves into the stent cavity.

These and other objects will be apparent to those skilled in the art based on the teachings herein. Other objects and advantages of the present invention will become apparent from the following description and accompanying drawings.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 is an illustration of one way the in-stent restenosis device would be used on a patient.

FIG. 2 shows current stents that will function as microwave scattering devices.

FIG. 3 shows a possible embodiment for scatterers with broken symmetry.

FIG. 4 shows an alternative embodiment using a shield to block e-field penetration into the stent cavity.

FIG. 5 shows an embodiment where a diode is placed in the gap.

FIG. 6 is a schematic showing the key components in the microwave sensor electronics.

FIG. 7 shows a flow chart illustrating the key elements of the system control software.

### DETAILED DESCRIPTION OF THE INVENTION

An object of the present invention is to provide a device and method to evaluate in-stent restenosis. This invention utilizes a microwave device to monitor occlusions within implanted stents and provide information that can be used by the patient and the physician to determine the best course of treatment for the patient

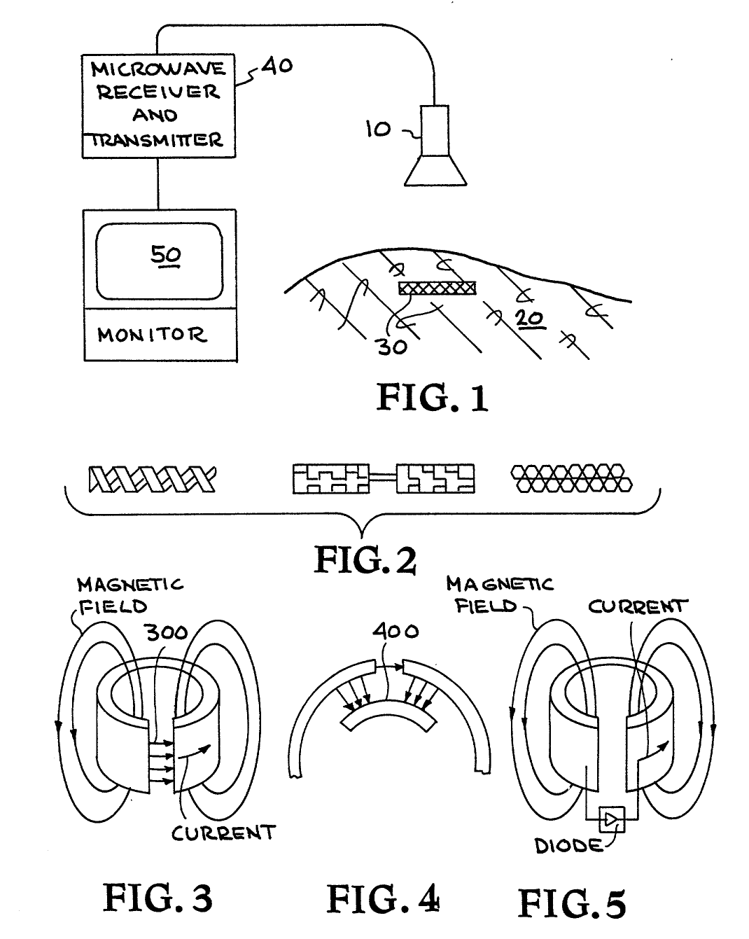

FIG. 1 shows an embodiment of the present invention in normal use. A microwave transmitter/receiver 10 is placed near the chest of the patient 20 and transmits microwaves of selected frequency toward the implanted stent 30. A microwave receiver 10 detects the microwaves scattered from the stent 30. For purposes of this disclosure, the term “scattered” includes the term “reflected” as well as the term “scattered”. In one embodiment, the microwave receiver may be separate from the transmitter so that it can be located either near the transmitter or moved around the patient to detect the scattering amplitude as a function of angle. The control module 40 varies the transmitted microwave frequency over a selected range (e.g., 0.1-20 GHz) and records the detected scattered microwave signal. This information is processed by a microprocessor and displayed on a monitor 50. The control module can perform similar measurements for multiple microwave polarizations and for different transmitter-receiver orientations. The data collected with this system for each patient immediately after stent implant is stored by the microprocessor. On a regular basis (e.g., every month) the patient returns to the doctor to have the complete set of microwave measurements repeated. The new measurements are compared by the microprocessor to the original measurements collected after stent implant. If the change in signal indicates a significant change the patient is scheduled for additional tests (e.g., angiography).

FIG. 2 shows currently used stents that will function as microwave scattering devices whose scattering frequency and amplitude will change with restenosis.

Although this technique is applicable to existing metallic stents, alternative stent designs can enhance the scattered signal and exhibit a narrow resonance frequency that can be more sensitive to plaque formation. For example, FIG. 3 shows a different stent design where the stent has a gap 300 along the cylindrical axis. This stent behaves as a high Q circuit simply through breaking the cylindrical symmetry. In this case the higher Q provides enhanced discrimination. Further, because the electric field is highly concentrated at the gap, this embodiment will be ultra-sensitive to small changes in dielectric properties near the gap, and enables restenosis detection at a very early state.

For some embodiments the signal variation with the simple gap design shown in FIG. 3 will be too large. In this case a shield 400 in FIG. 4 can be used to block e-field penetration into the stent cavity. In an alternative embodiment the gap can be filled with dielectric material to tune the sensitivity of the system to plaque buildup. Also a filled gap reduces the risk of tissue herniating through the gap.

For some embodiments, the simple gap shown in FIG. 5 will be replaced by a semi-conductor diode as shown. The non-linear response with microwave amplitude which results from the non-linear impedance of the diode generates harmonics of the fundamental microwave frequency which will be easier to measure against the scattering background from tissue interfaces which will all be at the fundamental frequency of the microwaves.

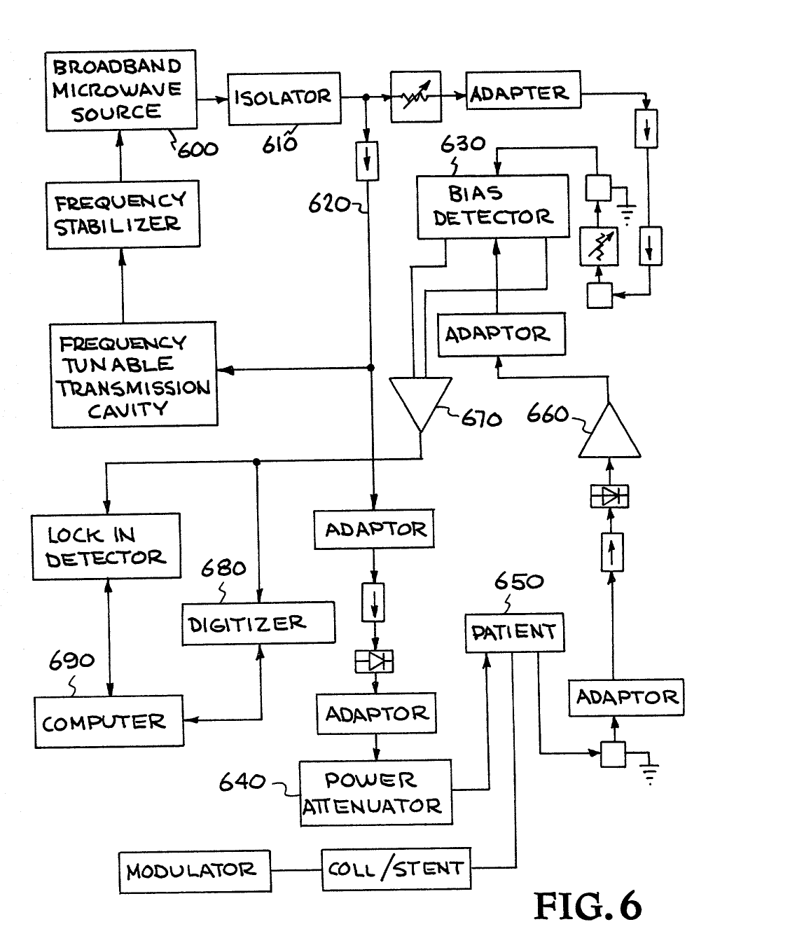

FIG. 6 is a block diagram showing the key components of the microwave transmitter and detection system. A broadband microwave source 600 passes through an isolator 610 and a small amount of power is split off 620 to monitor and stabilize the microwave source 600. The microwave source 600 is further split to bias detectors 630. The rest of the power is attenuated 640 and then transmitted to the patient 650. The power is focused on the patient with a transmitter/receiver. The stent scatters the microwave field and the scattered intensity is collected by the transmitter/receiver. This scattered intensity is amplified initially with a GaAs FET preamplifier 660, detected with diodes biased into their linear range 630, differentially amplified 670, and then digitized 680 and stored on a computer 690 for processing. Microwave polarizers can be used to polarize the transmitted signal and detect the scattered intensity as a function of polarization. Output power for this Network system (20 dbm) may need to be enhanced with additional amplifier and associated components to isolate receiver and circulate signal. This detection sensitivity is −110 dbm.

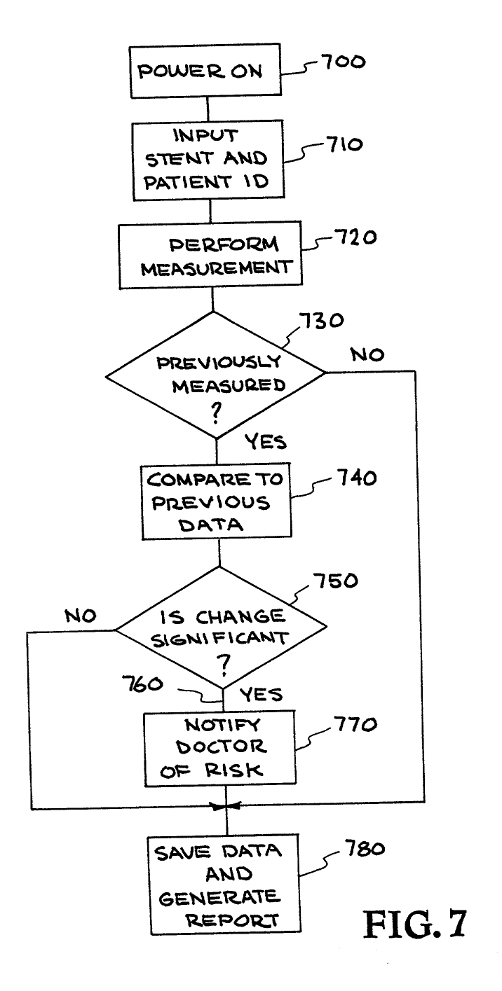

FIG. 7 shows a flow chart illustrating the key elements of the system control software. When first powered on (700) the system performs a self-test to verify that the system is operating correctly. If the system is operating correctly the computer asks the user for the stent type and patient ID (710) and whether a baseline reading for this patient already exists. The software then initiates a complete measurement (720) and records the data. The system then determines whether this is a first measurement or if previous data exists. If this is a baseline record (730), the software simply analyzes the data and verifies that signal levels and frequency range are suitable. If previous records exist 740 for this patient the software then performs an analysis 750 of all the data to determine whether a dangerous change or trend exists that indicates possible restenosis 760. The results of the analysis are presented (e.g., to the doctor) (770) to enable interpretation of the data to provide a diagnosis. The data is saved and a report is generated 780.

The data collected by the system will be the scattered intensity as a function of frequency. In the analysis section the characteristic peaks in intensity are identified using standard peak detection algorithms well known in the art. The frequency of the peaks will be compared to the baseline measurement to calculate the frequency shifts. It is the magnitude of these frequency shifts that determine the extent of restenosis. A shift of 1-5% would indicate significant restenosis. If resonance peaks are broad then a full spectral comparison between baseline and new data can be performed to calculate and difference index. Alternative analysis techniques could include neural networks that are trained with early clinical data.

In an alternative embodiment a compact microwave transmitter and receiver with reduced frequency range (or even single frequency) and options can be provided to the patient and used at home to monitor stent condition on a daily or weekly basis. This compact unit would be pretuned after the operation to operate near the resonant frequency of the stent. As restenosis occurs, the detected signal amplitude decreases and if it decreases below a preset amplitude an alarm would sound. The alarm signal level is a function of stent type and the shape of the patient. In another embodiment, a selected individual or group such as a health care professional or organization could be automatically notified by wireless or hardwired technology that the preset amplitude had been reached or exceeded.

In yet another embodiment modulated electromagnetic radiation are used to excite acoustic oscillations in the stent. An ultrasound transducer that is placed in contact with the skin detects these acoustic oscillations. Existing ultrasound transducers or imaging systems could be used to detect the oscillations. By using pulsed or modulated EM radiation, and time gated ultrasound detection the signal to noise of the system can be significantly increased. Conventional lock-in amplifier techniques could also be employed to detect the ultrasound, which has the characteristic frequency of the modulated EM. For optimum detection the ultrasound will be in the range of 200 kHz to 2 MHz.

Although described for cardiovascular stents this technique can be applied to all applications where stents are used. This includes neurovascular stents, and stents used for urological applications.

The above descriptions and illustrations are only by way of example and are not to be taken as limiting the invention in any manner. One skilled in the art can substitute known equivalents for the structures and means described. The full scope and definition of the invention, therefore, is set forth in the following claims.

We claim:

1. An apparatus, comprising:
a stent; and
means for enhancing microwave radiation that is scattered from said stent, wherein said means produces a larger scattered microwave radiation field over that which would occur from said stent absent said means.

2. The apparatus of claim 1, wherein said means comprises a cylindrical symmetry variation in said stent.

3. The apparatus of claim 1, wherein said stent comprises a cylindrical axis, wherein said means comprises a gap along said cylindrical axis.

4. The apparatus of claim 1, further comprising a microwave transmitter for transmitting microwave radiation to said stent, wherein said stent produces scattered or reflected microwave radiation.

5. The apparatus of claim 4, further comprising a microwave receiver for receiving data comprising said scattered or reflected microwave radiation.

6. The apparatus of claim 5, further comprising computer hardware with software comprising an algorithm for analyzing said data to determine whether in-stent restenosis has occurred.

7. The apparatus of claim 6, wherein said software further comprises an algorithm for analyzing said data to quantify the amount of in-stent restenosis that has occurred.

8. The apparatus of claim 3, further comprising dielectric material within said gap.

9. The apparatus of claim 3, wherein said stent comprises a stent cavity, said apparatus further comprising an electric shield to block electronic field penetration into said stent cavity.

10. The apparatus of claim 1, wherein said means comprises a dimension that is tuned to maximize the detection of in-stent restenosis.

11. The apparatus of claim 1, wherein said means comprises a dimension that is tuned for at least one microwave frequency.

12. The apparatus of claim 3, wherein said gap comprises a dimension that is tuned for at least one desired frequency.

13. The apparatus of claim 1, wherein said stent comprises a compact state with a first cross-sectional area and an expanded state with a second cross-sectional area that is greater than said first cross-sectional area.

14. The apparatus of claim 1, wherein said stent is selected from the group consisting of a cardiovascular stent, a neurovascular stent and a urological stent.

15. The apparatus of claim 6, further comprising an alarm, wherein said algorithm triggers said alarm if in-stent restenosis is present.

16. The apparatus of claim 7, further comprising an alarm, wherein said algorithm triggers said alarm if in-stent restenosis exceeds a pre-set level.

17. The apparatus of claim 7, further comprising an alarm, said apparatus further comprising a wireless transmitter operatively connected to said computer hardware, wherein said algorithm notifies a selected contact if in-stent restenosis exceeds a pre-set level.

18. An apparatus, comprising:
a stent; and
a microwave transmitter or microwave receiver operatively connected to said stent, wherein said microwave transmitter is configured for transmitting microwave radiation to said stent to produce scattered microwave radiation, and wherein said microwave receiver is configured for receiving data comprising microwave radiation scattered from said stent.

19. The apparatus of claim 18, further comprising computer hardware with software comprising an algorithm programmed to perform a task selected from the group consisting of (i) analyzing said data to determine whether in-stent restenosis has occurred and (ii) analyzing said data to quantify the amount of in-stent restenosis that has occurred.

20. The apparatus of claim 18, further comprising means for enhancing microwave radiation that is scattered or reflected from said stent, wherein said means produces a larger scattered or reflected microwave radiation field over that which would occur from said stent absent said means.

21. The apparatus of claim 20, wherein said means comprises a cylindrical symmetry variation in said stent.

22. The apparatus of claim 18, wherein said stent comprises a cylindrical axis, wherein said means comprises a gap along said cylindrical axis.

23. The apparatus of claim 22, further comprising dielectric material within said gap.

24. The apparatus of claim 20, wherein said stent comprises a stent cavity, said apparatus further comprising an electric shield to block electronic field penetration into said stent cavity.

25. The apparatus of claim 20, wherein said means comprises a dimension that is tuned to maximize the detection of in-stent restenosis.

26. The apparatus of claim 20, wherein said means comprises a dimension that is tuned for at least one microwave frequency.

27. The apparatus of claim 22, wherein said gap comprises a dimension that is tuned for at least one desired frequency.

28. The apparatus of claim 18, wherein said stent comprises a compact state with a first cross-sectional area and an expanded state with a second cross-sectional area that is greater than said first cross-sectional area.

29. The apparatus of claim 18, wherein said stent is selected from the group consisting of a cardiovascular stent, a neurovascular stent and a urological stent.

30. The apparatus of claim 19, further comprising an alarm, wherein said algorithm triggers said alarm if in-stent restenosis is present.

31. The apparatus of claim 19, further comprising an alarm, wherein said algorithm triggers said alarm if in-stent restenosis exceeds a pre-set level.

32. The apparatus of claim 19, further comprising an alarm, said apparatus further comprising a wireless transmitter operatively connected to said computer hardware, wherein said algorithm notifies a selected contact if in-stent. restenosis exceeds a pre-set level.

33. An apparatus, comprising:
a computer readable medium; and
computer software programmed onto said computer readable medium, wherein said software comprises an algorithm programmed to perform a task selected from the group consisting of (i) analyzing data to determine whether in-stent restenosis has occurred and (ii) analyzing data to quantify the amount of in-stent restenosis that has occurred.

34. The apparatus of claim 33, wherein said algorithm is programmed to perform the task of analyzing data to determine whether in-stent restenosis has occurred, said algorithm comprises the steps of:
analyzing measurement of scattered signal as a function of microwave frequency to identify peaks; and
comparing peaks to previously measured baseline when no restenosis had occurred, wherein if the shifts in the peak locations exceed a predefined maximum then in-stent restenosis has occurred.

35. The apparatus of claim 33, wherein when said algorithm is programmed to perform the task of analyzing data to quantify the amount of in-stent restenosis that has occurred.

36. A method for detecting in-stent restenosis in a patient that has an implanted stent, comprising:
probing said stent with microwave radiation to produce reflected or scattered microwave radiation;
detecting and collecting said reflected or scattered microwave radiation to produce data; and
analyzing said data to determine whether in-stent restenosis has occurred.

37. A method for quantifying the amount of in-stent restenosis that has occurred in a stent implanted in a patient, comprising:
probing said stent with microwave radiation to produce reflected or scattered microwave radiation;
detecting and collecting said reflected or scattered microwave radiation to produce data; and
analyzing said data to quantify the amount of in-stent restenosis that has occurred in said stent.

38. The method of claim 37, wherein the microwave transmitter and receiver have a fixed relative position.

39. The method of claim 37, further comprising optimizing the signal levels of said reflected or scattered microwave radiation by moving at least one of a microwave transmitter or a receiver.

40. The method of claim 37, wherein said microwave radiation comprises less than 200 MHz to excite acoustic oscillations in said stent, wherein the step of detecting is carried out with an ultrasound transducer placed in contact with the skin to detect acoustic oscillations.

41. The method of claim 40, further comprising pulsing said radiation and wherein said step of detecting includes time gated ultrasound detection to increase signal to noise.

42. A method for preparing a patient for detection of in-stent restenosis, comprising implanting a stent within a patient, wherein said stent comprises means for enhancing microwave radiation that is scattered reflected from said stent, wherein said means produces a larger scattered or reflected microwave radiation field over that which would occur from said stent absent said means.

43. The apparatus of claim 3, further comprising a microwave diode within said gap.

44. The apparatus of claim 5, wherein said microwave transmitter and said microwave receiver operate over a frequency range of 0.1 to 50 GHz.

45. The apparatus of claim 5, wherein said microwave transmitter and said microwave receiver are polarization sensitive.
